(19) United States
(12) Patent Application Publication
(10) Pub. No.: US 2017/0014058 A1
(43) Pub. Date: Jan. 19, 2017

(54) INTERDIGITAL DETECTION SYSTEM

(71) Applicant: Steven C. White, (US)

(72) Inventors: Steven C. White, Las Vegas, NV (US); Charles V. Evans, JR., Santa Monica, CA (US)

(21) Appl. No.: 15/043,494

(22) Filed: Feb. 12, 2016

Related U.S. Application Data
(60) Provisional application No. 62/115,606, filed on Feb. 12, 2015.

(51) Int. Cl. A61B 5/1455; A61B 5/00; A61B 5/117; A61B 5/024; A61B 5/01; A61B 5/145; A61B 6/00

(52) U.S. Cl. CPC A61B 5/14552; A61B 5/14546; A61B 5/6825; A61B 5/6843; A61B 5/4845; A61B 5/14532; A61B 6/485; A61B 5/02438; A61B 5/01; A61B 5/117

(57) ABSTRACT

A system for detection of blood analytes comprising a detector, a light source, and a resilient connector extending between the detector and the light source and positioning the detector and light source relative to one another such that one is positioned on the dorsal surface of a user's interdigital space and the other is positioned on the palmar surface of a user's interdigital space.

### BACKGROUND OF THE INVENTION

This invention relates to the field of medical devices for non-invasive blood trace analyte detection, measurement, and medical analysis thereof. More specifically, the invention comprises a non-invasive measurement system using a detection mechanism positioned between the interdigital space targeting arterial blood supply.

### SUMMARY

The detection mechanism used in a device or system may be any of a wide variety of detection mechanisms, such as spectroscopy, fluoroscopy, pulse-oximetry, or microscopy. For example, the detection mechanism may be spectroscopy. Spectroscopy covers analysis of any blood trace analyte whereby the light source located on bottom (palmar aspect) side, emits electromagnetic radiation to pass through the arterial blood supply (palmar artery), and the detector (dorsal aspect) side receives the light information that has passed through the interdigital spacing between probe and detector. The form of spectroscopy or other detection mechanism method will be integrated into the system.

The form of spectroscopy may also cover the opposite direction of transmission whereby the light source located on the top (dorsal aspect) side, emits electromagnetic radiation to pass through the arterial blood supply (palmar artery), and the detector (palmar) side receives the light information that has passed through the interdigital spacing between probe and detector. With either form of spectroscopy, the method will be integrated into the system.

The method for non-invasively evaluating blood trace analytes of a subject may include providing a source of electromagnetic radiation to a device, positioning the device at an interdigital location between fingers or toes of a subject, and receiving the electromagnetic radiation that has passed through or reflected from the subject between the interdigital space between the proximal phalanx and between the adjacent metacarpophalangeal joints.

In an embodiment of the present invention, the method includes applying the device to a surface of the subject at a substantially constant pressure, and the device can be applied such that it is it to be substantially flush with the surface of the subject's skin. The device may also include a touch and/or other type of sensor to activate the device and/or a pressure sensor to ensure the device is operated at a particular pressure. The device may or may not include a pulse LED or electromagnetic transmission device to ensure proper placement, for accurate detection and measurement of arterial blood.

In an embodiment of the present invention, the system includes a probe light source base (positioned at the palmar aspect between the metacarpophalangeal joint) and a probe detector head (positioned at the dorsal aspect between the metacarpophalangeal joint). The system may include foam-like padding (e.g. neoprene for optimal ergonomic fit) which also serves to block any unnecessary light at the interface.

In an aspect of an embodiment of the present invention, the electromagnetic radiation can be near infrared radiation, fluoroscopy, pulse oximetry, and/or transmission spectroscopy methods. A wide variety of light sources may be used in the device as appropriate to the form of detection being implemented.

In another embodiment to the present invention, an apparatus for non-invasively evaluating blood trace analytes of a subject includes a means for providing a source of electromagnetic radiation to a device, a means for positioning the device at an interdigital location between fingers or toes of the subject, and a means for receiving the electromagnetic radiation absorbed or reflected from the subject. Receiving the electromagnetic radiation may or may not occur on the dorsal aspect of the interdigital space, whereby the light source may or may not be positioned on the palmar side, and emits electromagnetic radiation.

Another embodiment of the present invention is directed toward an interdigital detection light system for non-invasively evaluating body fluids of a subject. The system includes a source of electromagnetic radiation, a probe light source which may or may not include fiber optics conveying electromagnetic radiation from the source to the probe detector, which may or may not include fiber optics, adapted to receive the emitted electromagnetic radiation at an interdigital location between fingers or toes of the subject. The light source transmits light from the palmar aspect between two metacarpophalangeal joint, and the detector receives information from the dorsal aspect between two metacarpophalangeal joints.

A wide variety of detectors or detection mechanisms may be used in the device as may be appropriate to the analyte to be detected. Various infrared detectors may be used to detect blood alcohol or blood glucose levels for example. Various imaging mechanisms, whether two dimensional or three dimensional, may also be used.

In another embodiment of the present invention, the detection light system includes a source of electromagnetic radiation that detects near infrared radiation, X-ray (fluoroscopy), pulse-oximetry, or any wavelength capable of transmission spectroscopy.

### BRIEF DESCRIPTION OF THE DRAWINGS

FIG. 1a is a schematic diagram of an embodiment of the present invention.

FIG. 1b is a schematic diagram of another embodiment of the present invention.

FIG. 2 is a schematic rendering of one embodiment of a system of the present invention.

FIG. 3 is a schematic rendering of another embodiment of a system of the present invention.

FIG. 4 is a schematic rendering of another view of the embodiment shown in FIG. 3.

FIG. 5 is a diagram showing blood pathways in areas of potential interest for the present invention.

### DETAILED DESCRIPTION

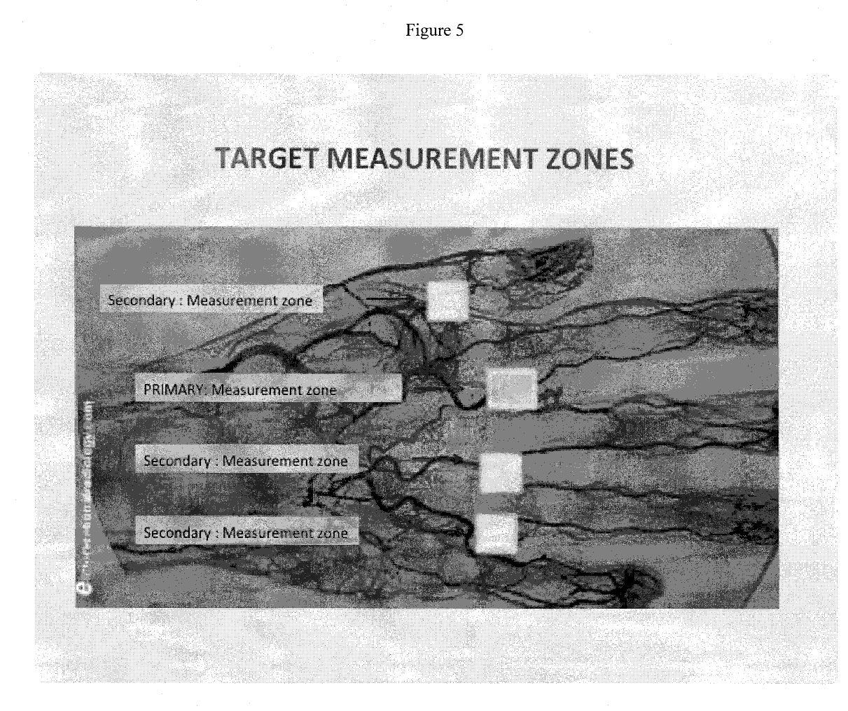

An apparatus for evaluation of a subject's body fluids may be used at the interdigital region adjacent to or in between a subject's extremities. The evaluation positioning consists of a light source (probe) on the palmar aspect, which may or may not include fiber optics, and a detector (probe) on the dorsal aspect, which may or may not include fiber optics. The measurement device may be positioned flush with skin at both palmar/dorsal aspects of hand between the second and third proximal phalanx and between the (MCP) metacarpophalangeal joint. The spectroscopy, fluoroscopy, pulse-oximetry and/or microscopy targets the palmar digital arterial blood supply at the interdigital spacing between any hand or foot digit, see FIG. 5., and can detect and quantify multiple blood analytes using various methods. The system can be used measure blood trace analytes such as: blood alcohol, blood glucose, cancer biomarkers, biomarkers, chemotherapy, pharmaceuticals drugs, blood oxygenation levels, and or pulse/heart rate for any medical screening or diagnostic purposes.

The device may possess one or more of the movements such as rotational, translational, and/or vertical freedom necessary for the probes to contact the subjects tissue at a consistent angle and pressure while accommodating the different size of the subjects extremities, and may be of any memory yielding material optimized for attaining reproducible blood flow to the region of the subject that is measured, and for minimizing the effects of pulling, stretching, pressing, compressing the subject's skin. A smooth fit that does not clamp or clip to the user is preferred.

To allow the detector to rest flush with the user's skin surface, a pivot hinge 24 may be provided at the sides of the detector facing the adjacent user digits. The pivot hinge on each side is received into openings in a connector or carrier means 14 so that the detector can rotate as needed to rest flush.

In addition, the measurement device may be coupled with a temperature measurement means that detects the subject's body temperature in or near the region being measured, or the subject's core or mean body temperature, or the ambient temperature proximate to the probes, detectors, or sensors.

In addition, the spectroscopic measurement may be coupled with a biometric scanner that detects the subject's identity. the biometric scanner can be positioned to measure the fingerprint of any digit on the hand or foot. The system may couple blood trace analyte measurements with GPS, accelerometer, or gyroscopic measurements: speed, velocity, global positioning, altitude, longitude position, latitude position, or relative location. The system may couple heart rate (pulse), and blood oxygenation measurement levels, with GPS or gyroscopic measurement. The system may also include one or more methods to communicate information of a subject to a mobile device, computer, cell phone, subject database, or computer software.

An apparatus for non-invasive spectroscopic measurement according to the one embodiment of the present invention is suitable for many applications, particularly for non-invasively evaluating blood trace analytes such as: synthetic markers or nano-particles that have been coupled to certain components or analytes in a bodily fluid of a subject, such as the subject's blood, where the markers or nano particles are designed to have a particular spectroscopic or visual signature. In addition, the apparatus could be used to detect toxins or hazardous chemicals in the blood. The apparatus can measure cancer biomarkers, pulse (heart rate), blood oxygenation, and pharmaceutical drugs.

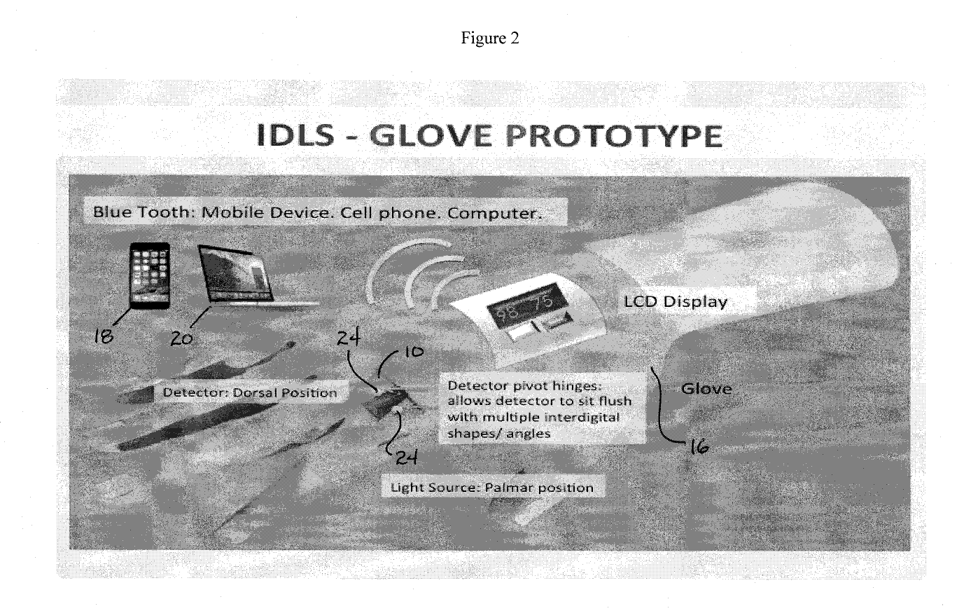

Such an apparatus could be useful in biomedical applications. For example the apparatus could be used to monitor cancer biomarkers, or biomarkers in the blood for preventative screening. In such applications, the apparatus could be configured to be attachable to a patient for continuous monitoring whereby the system is integrated into a glove. See FIG. 2. When coupled with a biometric identification device, the apparatus could also link to insurance and/or medical records of a patient. This link could be used to update patient information or to make comparisons of past fluoroscopic readings, by way of example, as an aid in diagnosis and treatment of patients with cancer.

Such an apparatus could be useful in the biomedical application of heart rate and blood oxygenation monitoring and serve as a pulse oximetry sports glove. When coupled with GPS, and accelerometer, or gyroscopic device, the apparatus can give real-time information on heart rate and blood oxygenation level with body position. This technology can transmit via Bluetooth, or direct sync to a mobile device, cell phone, or computer.

Multiple embodiments include many different light sources to provide a source of electromagnetic radiation. In one embodiment, fluoroscopy-imaging technique using x-rays can be used to measure cancer biomarkers or blood trace analytes in two dimensional or three dimensional capacity. In another, embodiment, a quartz halogen lamp is used to provide a source of electromagnetic radiation in the near infrared region; suitable for non-invasive measurement of concentrations of blood components or blood analytes, such as alcohol or glucose. Other light producing devices such as flash lamps, tungsten-halogen lights, light emitting diodes, quartz halogen, or laser sources can be used in conjunction with filtering mechanisms to produce a certain spectral range that corresponds to the spectral range absorption of other targeted tissue components or analytes to be measured. The transmission of this electromagnetic radiation from the palmar aspect at the interdigital space between the metacarpophalangeal (MCP) joint and received on the dorsal aspect of the interdigital space between the metacarpophalangeal (MCP) joint are part of what makes this invention unique. Additionally, the system may or may not integrate into a glove to ensure proper fit, placement, and ergonomic comfort at the target measurement zone. The detector pivot hinge is an additional design component that ensures optimal flush positioning of the detector and light source with the interdigital space. The system may or may not be connected to a spectrometer with or without fiber optics. The spectrometer may or may not include light source and detector at some aspect of the hand; either dorsal or palmar. The system may or may not be connected to LCD screens, which will display/process measurement metrics/information, which can transmit data via Bluetooth or direct connection to a computer, wireless device, or processing software system.

As noted, the detector or detector mechanism may be in the form of microscopy, or other form of imaging, whether two dimensional or three dimensional. For example, the detector may be a microscope on a chip. Such chips are available from the Swiss company Nanolive, and are described in U.S. Pat. No. 8,937,722, the entire disclosure of which is incorporated by reference herein.

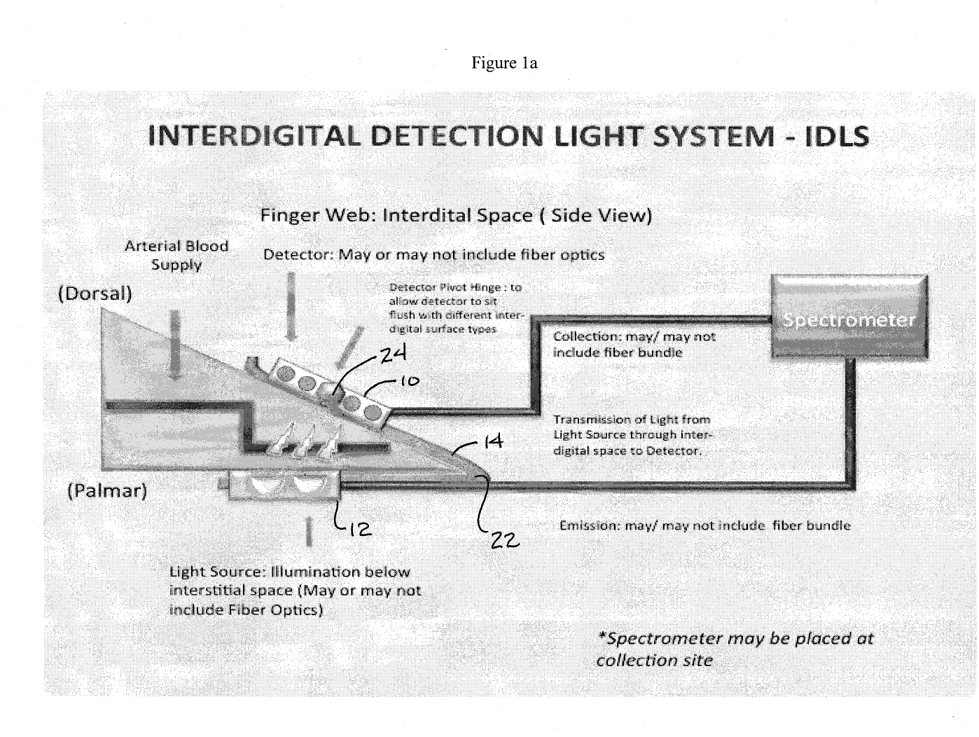

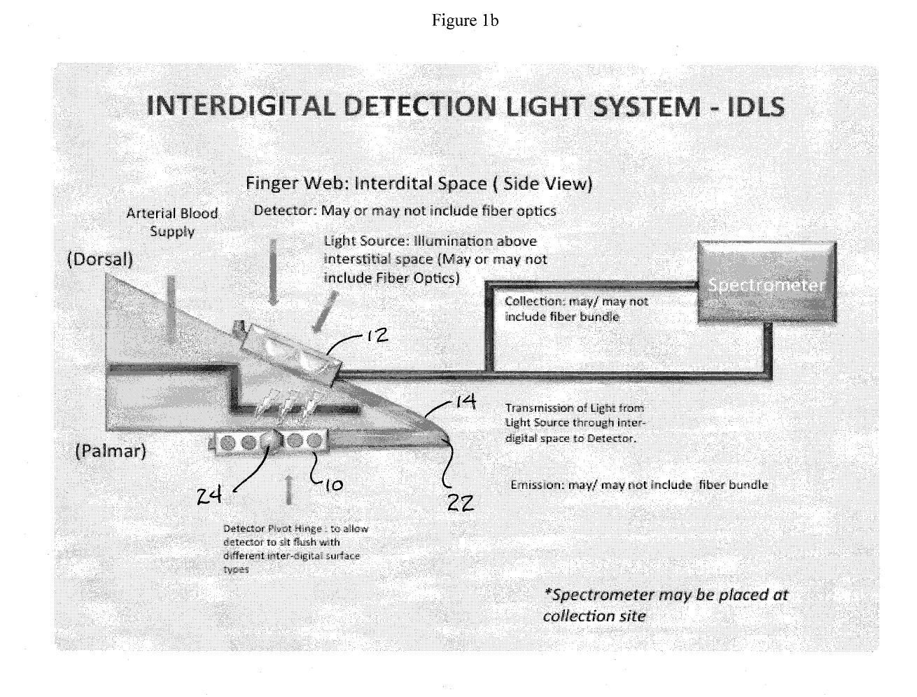

FIGS. 1a and 1b illustrate a schematic side view of the system interface having a probe head (detector) 10, which may or may not include fiber optics, a probe base (light source) 12 that may or may not include fiber optics, and a mechanism 14 connecting the probe head to the probe base. The connection may include any material that allows the light source and detector to sit flush with the interdigital space. The light source will emit from the palmar aspect with detection on the dorsal aspect positioned between any interdigital space of the hand, and or the interdigital space of the feet at the palmar digital crease. The light source may also emit from the dorsal aspect with detection on the palmar aspect positioned between any interdigital space of the hand, and or the interdigital space of the feet at the palmar digital crease. The detector and light source must be flush with skin and are ergonomically positioned using the interface. The method of spectroscopy, fluoroscopy, pulse-oximetry, or microscopy covers any form of electromagnetic radiation at any wavelength and any type of detector whereby the light source is positioned on the palmar or dorsal aspect, and the detector is correspondingly positioned on the dorsal or palmar aspect of the subject.

The light source and/or the detector and/or any collector mechanism used in the system may be incorporated into or on a chip. As noted, various different light sources, detectors and collectors may be used with the system depending upon the analyte being monitored. In an embodiment of the system, the system may be configured to accept and retain interchangeable chips. In this way, a system may be customized for a particular patient, situation or analyte by interchanging the chips used in the system. The light source chip 12 may be changed to incorporate a different light source. Similarly, the detector or collector chip 10 can be changed as is appropriate to the circumstances. The light source and detector chips may also be interchanged between dorsal and palmar aspects if desired. Removeable chips also allow for removal and replacement of light sources and/or detectors that have stopped operating.

FIG. 2 illustrates a method of spectroscopy (sensor) integrated into a glove 16 where the system is stitched into a glove, which serves as the mechanism that connects the light source probe and detector. The system may also include neoprene padding to ensure no peripheral light into a measurement zone. Light source can emit from either palmar/dorsal aspect, and detection can occur at either dorsal/palmar aspect.

Measurement site occurs at the interdigital space between the first (index) finger and the second (middle) finger, but is not limited to this region and can include any interdigital space whereby arterial blood supply is targeted for detection, measurement, and or analysis of measureable blood trace analytes. See FIG. 5. The system also illustrates the ability to communicate subject information to a mobile device 18, cell phone, computer 20, patient database, or computer software. Communication of information can occur via direct connectivity (e.g. USB) or via Bluetooth. Biometric identification capabilities are also possible to exclusively identify patient, and ensure HIPAA compliance. Spectroscopy measurements and data gathered can couple with GPS or gyroscopic measurements.

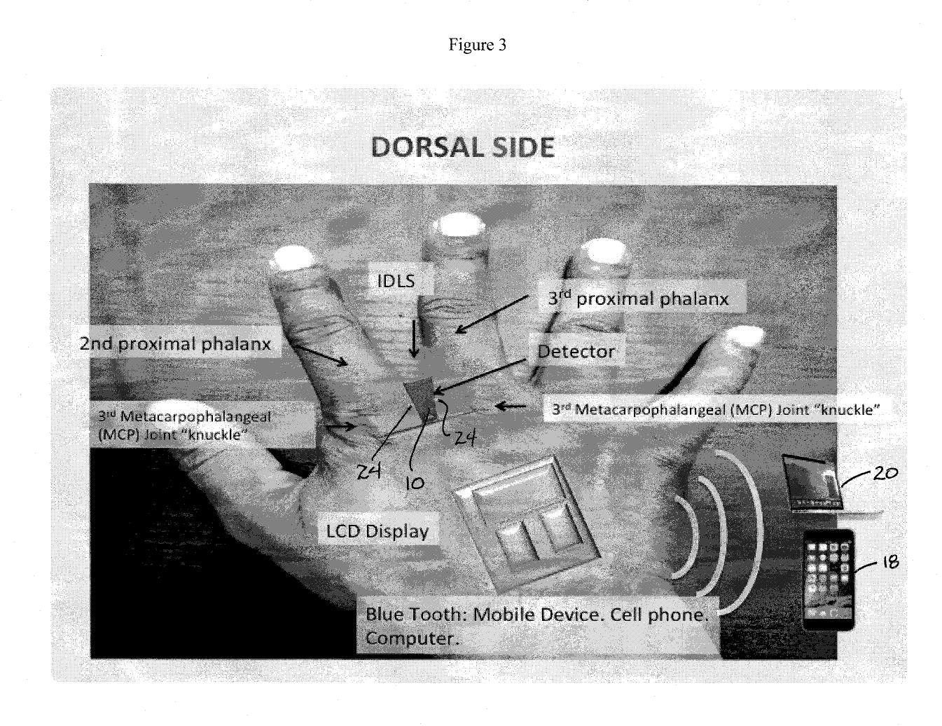

FIG. 3 illustrates the top view of hand (dorsal) system location: Between the knuckles of any interdigital spacing whereby the system targets arterial blood supply for detection, measurement, and medical analysis of blood analytes measured at this site. This view illustrates the location and method of spectroscopy whereby the detector probe 10 collects from the dorsal aspect. The illustration is an artistic rendering of a system without the glove to demonstrate target measurement zone and ergonomics. The collection (detector) may or may not include a fiber bundle.

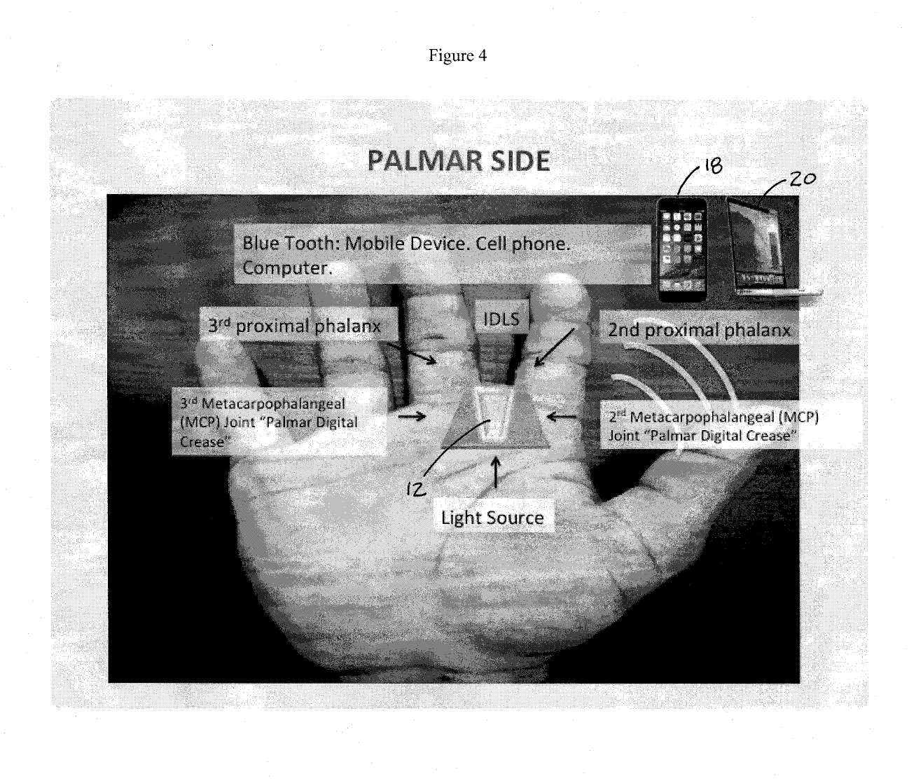

FIG. 4 illustrates the bottom view of the hand (palmar) system location: Between the palmar beds of any interdigital spacing whereby the system emits electromagnetic radiation directly through arterial blood supply for detection, measurement, and medical analysis of blood analytes at this site. This view illustrates the location and method of spectroscopy whereby the light source probe emits from the palmar aspect. The illustration is an artistic rendering of what the system will look like from the palmar aspect. The light source probe may or may not include a fiber bundle.

FIG. 5 illustrates the target measurement zones “TMZs” (arterial blood supply). The TMZ is the arterial blood supply from the digital palmar artery. The TMZ is at the interdigital space between the metacarpophalangeal joint.

The system will typically consist of a light source and detector made of any resilient material that allows for flush skin contact of both the detector and light source. This may include, but is not limited to: a specially designed tissue interface with an integrated detector and light source, spectrometer with/without fiber optics, fluoroscopy, X-ray, pulse oximetry, or microscopy.

The detector may or may not include fiber bundle. The detector/sensor can be a probe, collection fiber bundle, or chip sensor. The detector may be connected to a spectrometer via fiber bundle.

The light may or may not include fiber bundle. The light source can be any electromagnetic radiation: a quartz halogen, laser, light emitting diode, or a probe at the tip of light source fiber optics.

The interdigital tissue interface may consist of any malleable material that allows consistent pressure of the detector and light source to position flush with the interdigital space between the hand or feet digits. The interdigital tissue interface can be integrated into a glove, hand cradle, adjustable attachment means, etc.

The system interface components can be made of any resilient material with memory, and may include but not limited to: plastic, aluminum, carbon, rubber, latex, fabric, neoprene, etc.

The system may include a spring hinge 22 to facilitate constant pressure at the interdigital space.

The system can couple with biometric identification capabilities to share information with handheld devices, mobile phones, computers, patient databases or any software integrated into the aforementioned devices capable of receiving such.

The system can couple with global positioning system to sync information gathered from non-invasive blood analysis to location coordinates and subject positioning (e.g. heart rate at a specific location).

The system can couple with an accelerometer to facilitate battery preservation when sensor is not in motion for a specific amount of time (e.g. heart rate monitoring turned off when stationary for more than an hour or heart rate at a certain speed).

The system can couple with gyroscopic technology to pair orientation with non-invasive blood analysis (e.g. heart rate measured laying down).

The system can couple with an altimeter to pair altitude with non-invasive blood analysis (e.g. heart rate at a certain altitude).

What is claimed is:

1. A system for detection of blood analytes comprising:
a detector;
a light source;
a resilient connector extending between the detector and the light source and positioning the detector and light source relative to one another such that one is positioned on the dorsal surface of a user's interdigital space and the other is positioned on the palmar surface of a user's interdigital space.
